(19) United States
(12) Patent Application Publication
(10) Pub. No.: US 2013/0168408 A1
(43) Pub. Date: Jul. 4, 2013

(54) Method and Apparatus for Preventing Disease Spread

(71) Applicants: Dariusz Kubicz, Chicago, IL (US); Gerard Kubicz, Lombard, IL (US)

(72) Inventors: Dariusz Kubicz, Chicago, IL (US); Gerard Kubicz, Lombard, IL (US)

(21) Appl. No.: 13/342,402

(22) Filed: Jan. 3, 2012

(51) Int. Cl. A61L 2/18

(52) U.S. Cl. 222/1; 222/175

(57) ABSTRACT

A device for preventing the spread of disease especially in healthcare and food handling facilities by making it very quick and easy to disinfect hands without having to take extra steps. A small, disinfectant-filled bottle, typically flat, that fits snugly to the waist on a belt, or attached to a pocket, allows almost instantaneous disinfecting of hands simply by reaching down and spraying a small amount of disinfectant fluid on the hand while walking, standing or performing just about any other task. Being generally light and smooth, the device resists hanging up on material or objects, and it can be worn by both male and female personnel. A concave valve prevents false spraying. When empty, the device can be disposed of without any messy refilling.

### BACKGROUND

1. Field of the Invention

The present invention relates to the prevention of the spread of disease and more particularly to a waist-worn dispenser of disinfectant for use by staff in hospitals, nursing homes and any other place where bacteria can be spread.

2. Description of the Prior Art

Doctors, nurses and other staff at healthcare facilities are continually cautioned to wash and disinfectant hands both for their own protection and to prevent the spread of disease. However, it is a well-known fact that medical and nursing staff is spread very thin at many facilities, and being many times in a hurry, these personnel often skip disinfecting hands between visits to different patients.

Restaurant employees and other food handlers are also required to wash their hands typically after using the restroom. However, other than that, they seldom wash.

In many cases, doctors, nurses and nurse assistants (CNAs) wear rubber gloves when handling patients or residents. While this may protect the doctor or nurse, unless the gloves are changed between patients, there is no protection to the patient. In fact, contaminated rubber gloves are known to be a major factor in spreading disease.

Many facilities have bottles or dispensers of disinfectant liquid located near nursing stations and the like. Also, personnel may have access to small bottles of hand alcohol (a solution of alcohol and a protective substance for the skin). Both of these solutions are not likely to be used when the staffer is in a hurry, especially if there is an emergency.

It is a simple fact of human nature—if something a person knows should be done is very convenient, they will do it, even if they are in a hurry. On the other hand, if it is difficult, or takes extra time and/or steps, they will not do it especially when they are in a hurry, even though they know it should be done. This is true of disinfecting hands in a healthcare facility or food handling location.

It would be especially advantageous if healthcare facility and restaurant staffs had a method of preventing the spread of disease by easily and often disinfecting the hands without having to take extra steps or interrupt the standard routine.

Prior art spray bottles are typically bulky and are subject to false spraying because of the typically exposed pumps. It would be advantageous to have a small spray bottle that can immediately disinfect hands that is constructed to avoid false spraying.

### SUMMARY OF THE INVENTION

The present invention relates to a method and apparatus for preventing the spread of disease especially in healthcare and food handling facilities by making it very quick and easy to disinfect hands without having to take extra steps. A small, disinfectant-filled typically flat bottle that fits snugly to the waist on a belt, strap or pin, or attached to a pocket, allows almost instantaneous disinfecting of hands simply by reaching down and spraying a small amount of disinfectant fluid on the hands while walking, standing or performing just about any other task. The dispensing button and dispensing orifice are positioned so that the device can be operated by one hand, spraying that same hand. This can easily done between visits or contacts between each patient or resident, and it can be done without having to detour or interrupt the standard routine. Being generally flat, the device matches the body contour and is not bulky. Being generally light and smooth, it resists hanging up on material or objects, and it can be worn by both male and female personnel. The present invention has a specially constructed concave valve to prevent false spraying. When empty, the device can be totally disposable without any messy refilling. Healthcare, food handling and other facilities can stock cartons of this disposable and can require all staff to wear and use it.

### DESCRIPTION OF THE FIGURES

Attention is not directed to several drawings that are presented to illustrate features of the present invention:

FIG. 1 shows a front perspective view of an embodiment of the invention.

FIG. 2 shows the embodiment of FIG. 1 spraying disinfectant.

FIG. 3 is a back view of the embodiment of FIGS. 1-2.

FIG. 4 shows an embodiment of the invention being worn on a belt.

Several drawings and illustrations have been presented to aid in understanding the present invention. The scope of the present invention is not limited to what is shown in the figures.

### DETAILED DESCRIPTION

The present invention relates to a small, typically flat, plastic, spray bottle of hand disinfectant that can be worn on the belt or pocket for immediate and simply disinfecting of the hands. The disinfectant liquid can be any of those known in the art and similar to what is presently used in hospitals or other healthcare facilities. The bottle can be completely disposable to avoid messy refilling (optionally, the present invention can also be refilled, either on-site or by the manufacturer). A typical alcohol disinfectant has been shown to provide protection for the hands for around ninety minutes. The dispensing mechanism is designed to be operated by one hand spraying disinfectant on that same hand.

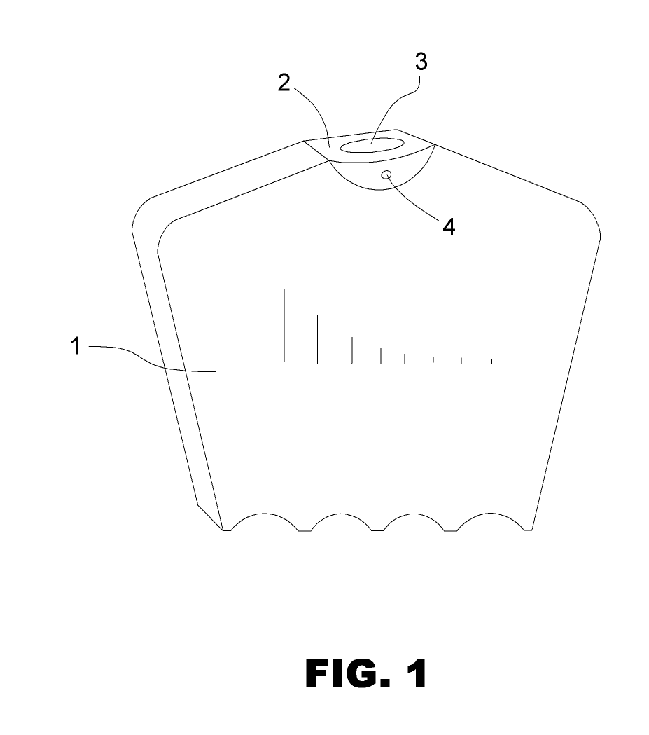

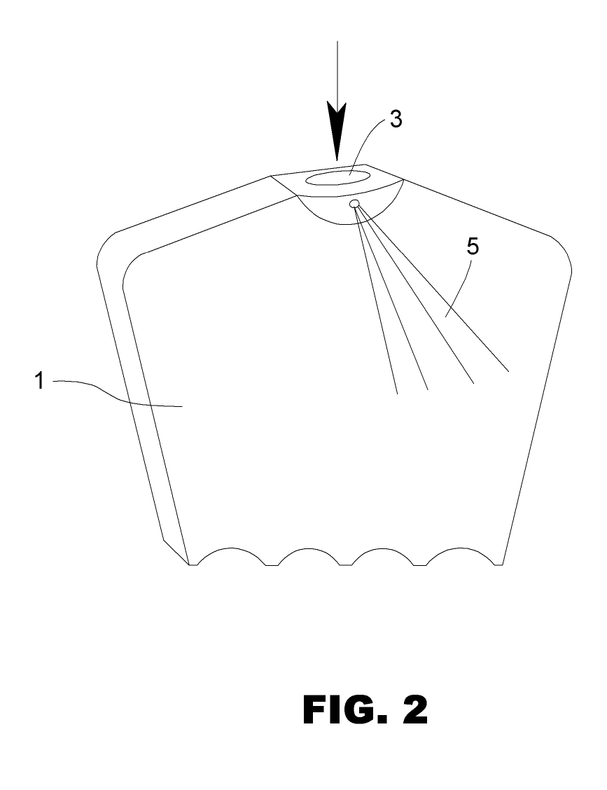

Turning to FIG. 1, an embodiment of the bottle 1 of the present invention can be seen. The bottle can be wider or narrower than shown as convenient. It can also optionally be slightly contoured to match the waist. The bottle 1 is typically filed with disinfectant fluid similar to that currently used in healthcare facilities. The bottle can hold any fluid. The top of the bottle 2 holds a small pump that can be activated with a push button 3 or by any other means to force a small stream of disinfectant out of an orifice 4 and onto the wearer's hands, or the contents of the bottle can under gas pressure. The top 2 and valve button 3 are constructed to be concave to prevent false spraying. FIG. 2 shows the process of pushing the button 3 with a resulting stream of disinfectant 5 that is typically directed forward of the wearer's body and onto the hand that is operating the device. Using the thumb, the user can push the button 3 with one hand spraying disinfectant on the same hand. The user then can rub the hands together sterilizing the hands. There is no need to further dry the hands, and there is no need to take extra steps to go to a disinfectant station.

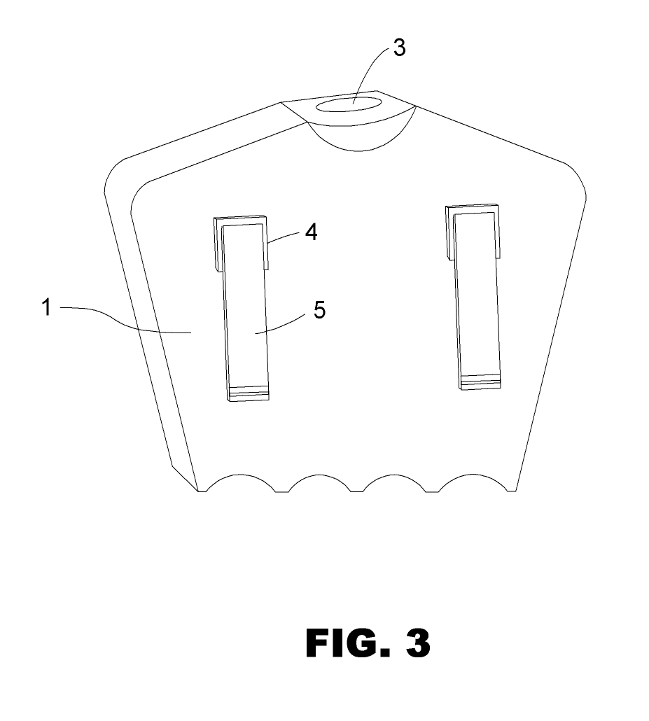

FIG. 3 shows back view of the embodiment of FIGS. 1-2. Attachments 4 hold a pair of belt clips 5 in place. The bottle 1 can simply be clipped to a belt or pocket or elsewhere on clothing. Alternative methods to attach the present invention to the person can be a clothing pin (for someone without a belt), a strap or any other way of attaching. The bottle 1 of the present invention can also be easily clipped to a shirt or pants pocket or a lab coat pocket. Any technique for attaching the bottle 1 to the wearer's clothing or belt is within the scope of the present invention.

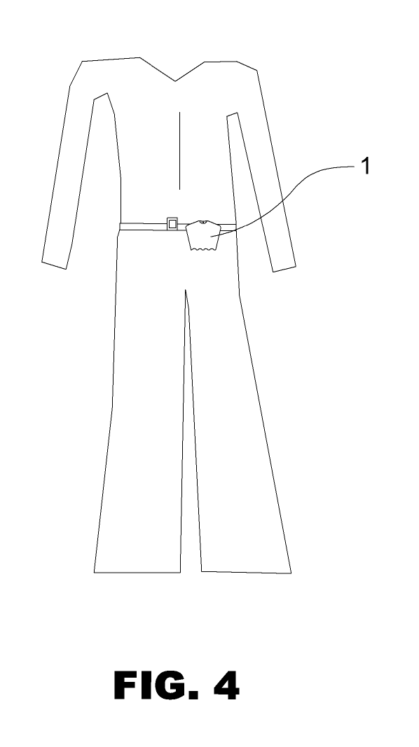

FIG. 4 shows a user wearing an embodiment of the present invention on their belt.

The bottle 1 of the present invention is preferably made of plastic; however any rigid material may be used and is within the scope of the present invention. The bottle 1 can optionally be supplied in different colors to match apparel and make it more attractive to wear or contain a message or logo on one or more of its surfaces.

In an alternate embodiment of the present invention, the top 2 can be sealed, and rather than being connected to an internal pump mechanism, the entire bottle can be under a small amount of pressure (typically by using a small quantity of pressurized gas or air). In this embodiment, the top 2 and button 3 simply actuate a valve that allows a pressurized gas to force the liquid out. A tube can extend from the cap top 2 internally to the bottom of the bottle 1.

While the device of the present invention is typically disposable and non-refillable, it is optionally possible to make the bottle 1 refillable by allowing the pump or dispensing unit 2 to be removed. This might be done upon recycling to the manufacturer.

While embodiments of the present invention have been described that are generally used in healthcare facilities and restaurants, it is within the scope of the present invention to use it in any context for any purpose as a spray bottle. In this broader usage, the present invention is very useful for cleaning windows, cars, furniture or anything that needs a spray. In these contexts, the liquid contained in the bottle can be simply sprayed onto a rag or towel.

The spray bottle of the present invention can optionally have signs, slogans or labels on its front or back side. This is particularly useful in, for example, a restaurant where food servers are wearing the bottles that announce that the server's hands are clean or that germs have been disinfected.

Several descriptions and illustrations have been presented to aid in understanding the present invention. One with skill in the art will realize that numerous changes and variations may be made without departing from the spirit of the invention. Each of these changes and variations are within the scope of the present invention.

We claim:

1. A method for preventing the spread of disease in a healthcare or food-handling facility comprising:
providing a flat, elongated spray bottle adapted to be worn on a belt or pocket by a user, said spray bottle having a concave region on an upper end with a dispensing button in said concave region and a spray orifice directing spray away from a wearer's body, wherein said dispensing button and said orifice cooperate to be operated by one hand and to spray disinfectant on the same one hand;
filling said spray bottle with disinfectant fluid;
allowing said spray bottle to be discarded when empty.

2. The method of claim 1 wherein said spray bottle is plastic.

3. The method of claim 1 wherein said bottle contains a pump mechanism.

4. The method of claim 1 wherein said bottle is under pressure.

5. An apparatus to permit easy disinfection of hands in healthcare facilities comprising:
a flat disinfectant spray bottle adapted to be worn on a belt or a pocket by a user, said spray bottle having a concave region on an upper end with a dispensing button in said concave region, said spray bottle also having a spray orifice directing spray away from a wearer's body, said dispensing button and orifice adapted to be operated by one hand spraying that same hand with disinfectant, wherein said spray bottle is non-refillable.

6. The apparatus of claim 5 wherein said spray bottle is plastic.

7. The apparatus of claim 5 wherein said spray bottle contains a pump mechanism.

8. The apparatus of claim 5 wherein said spray bottle is under pressure.

9. The apparatus of claim 5 further comprising a pair of belt clips on a rear surface of said spray bottle.

10. The apparatus of claim 5 wherein said spray bottle displays a message or logo.

11. A disposable disinfectant spray device comprising:
a bottle member containing disinfectant liquid;
a concave region on an upper end of said bottle member, said concave region containing a dispensing member;
an orifice on said bottle member positioned to cooperate with said dispensing member to be operated by one hand and to spray disinfectant on said one hand;
at least one attachment member adapted to secure said bottle member to a belt or a pocket.

12. The disinfectant spray device of claim 11 wherein said bottle member is configured to be either discarded when empty or refilled.

13. The disinfectant spray device of claim 11 wherein said bottle member is plastic.

14. The disinfectant spray device of claim 11 wherein said bottle member displays at least one message or logo.

15. The disinfectant spray device of claim 11 further comprising a dispensing pump mechanism.

16. The disinfectant spray device of claim 11 wherein contents of said bottle member are under pressure.

17. The disinfectant spray device of claim 11 wherein said bottle member is generally flat.

18. The disinfectant spray device of claim 11 wherein said attachment member includes a pair of belt clips.
